(19) United States
(12) Patent Application Publication
(10) Pub. No.: US 2022/0054325 A1
(43) Pub. Date: Feb. 24, 2022

(54) A SCALP PROTECTOR FOR USE FOLLOWING A HAIR TRANSPLANT PROCEDURE

(71) Applicants: Alik YASINOV, Haifa (IL); Yelena YASINOV, Haifa (IL)

(72) Inventors: Alik YASINOV, Haifa (IL); Yelena YASINOV, Haifa (IL)

(21) Appl. No.: 17/424,955

(22) PCT Filed: Jan. 23, 2020
(86) PCT No.: PCT/IB2020/050529

§ 371 Date: Jul. 22, 2021

Related U.S. Application Data
(60) Provisional application No. 62/795,602, filed on Jan. 23, 2019.

(51) Int. Cl. A61F 13/12; A61F 2/10

(52) U.S. Cl. CPC A61F 13/12; A61F 2013/00272; A61F 2/10

(57) ABSTRACT

The present invention provides a scalp protector for use following a hair transplant procedure conducted on a subject including: a cover having a central region and elongated portions extending from the central region for covering the surface of the scalp of the subject, each of the elongated portions provides at least one set of a first part of a snap fasteners closure; a band configured to extend around the periphery of the scalp of the subject, the band includes anchoring regions deployed on the band so as to align with the elongated portions of the cover, each of the anchoring regions providing at least one set of a complementary part of a snap fasteners closure for fastening to the elongated portions.

### TECHNICAL FIELD

The present invention is directed to hair transplant procedures.

### BACKGROUND OF THE INVENTION

Hair loss, also known as alopecia or baldness, refers to a loss of hair from part of the head or body. Hair loss occurs in more than 60% of men and in approximately 10% of women. Symptoms of hair loss include hair loss in patches usually in circular patterns, dandruff, skin lesions, and scarring. Hair loss is a major concern for many, and surgery to treat hair loss (i.e., hair transplantation) is the most common cosmetic surgery procedure performed today.

Hair transplantation is a surgical procedure, in which hair follicles from one part of the body, called the ‘donor site’, is transferred to a bald or balding part of the body known as the ‘recipient site’. The transplanted hair continues to grow in its new area throughout the patient's lifetime.

Though highly popular, hair transplantation procedures are prone to several unpleasant phenomena, such as, sensitive scalp, sore scalp, bruises, bleeding, etc. which may occur after the surgical procedure.

### SUMMARY OF THE INVENTION

The present invention provides a scalp protector for use following a hair transplant procedure.

Thus, according to an embodiment of the present invention there is provided a scalp protector for use following a hair transplant procedure conducted on a subject comprising: a cover having a central region and elongated portions extending from the central region for covering the surface of the scalp of the subject, each of the elongated portions provides at least one set of a first part of a snap fasteners closure; a band configured to extend around the periphery of the scalp of the subject, the band includes anchoring regions deployed on the band so as to align with the elongated portions of the cover, each of the anchoring regions providing at least one set of a complementary part of a snap fasteners closure for fastening to the elongated portions;

According to further features of an embodiment of the present invention, the band includes at least one set of a first part of a snapback closure on its proximal end and at least one set of a complementary part of a snapback closure on its distal end for fastening both ends of the band together.

According to further features of an embodiment of the present invention, the at least one set of a first part of a snapback closure on the proximal end of the band is a set of stud fasteners and the at least one set of a complementary part of a snapback closure on the distal end of the band is a set of socket fasteners.

According to further features of an embodiment of the present invention, the at least one set of a first part of a snapback closure on the proximal end of the band is a set of socket fasteners and the at least one set of a complementary part of a snapback closure on the distal end of the band is a set of stud fasteners.

According to further features of an embodiment of the present invention, the anchoring regions of the band include securing straps shaped to hold the elongated portions of the cover attached to the aligned anchoring regions.

According to further features of an embodiment of the present invention, the at least one set of a first part of a snap fasteners closure in the anchoring regions is a set of stud fasteners and the at least one set of a complementary part of a snap fasteners closure is a set of socket fasteners.

According to further features of an embodiment of the present invention, the set of stud fasteners and the set of socket fasteners have uneven numbers of fasteners so as to adjust the distance of the cover from the scalp of the subject.

According to further features of an embodiment of the present invention, the at least one set of a first part of a snap fasteners closure in the anchoring regions is a set of socket fasteners and the at least one set of a complementary part of a snap fasteners closure is a set of stud fasteners.

According to further features of an embodiment of the present invention, the set of socket fasteners and the set of stud fasteners have uneven numbers of fasteners so as to adjust the distance of the cover from the scalp of the subject.

According to further features of an embodiment of the present invention, the band includes removable pads or bandages on the inner surface of the band, the removable pads or bandages shaped so as to contact the subject's scalp.

According to further features of an embodiment of the present invention, the cover includes removable pads or bandages on the inner surface of the cover, the removable pads or bandages shaped so as to contact the subject's scalp.

According to further features of an embodiment of the present invention, the band includes a plurality of fragments at different distances in relation to the scalp of the subject.

According to further features of an embodiment of the present invention, the band is made of a semi rigid material.

According to further features of an embodiment of the present invention, the band is made of silicone.

According to further features of an embodiment of the present invention, the cover is made of a semi rigid material.

According to further features of an embodiment of the present invention, the cover is made of silicone.

### BRIEF DESCRIPTION OF THE DRAWINGS

Some embodiments of the present invention are herein described, by way of example only, with reference to the accompanying drawings. With specific reference to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

Attention is now directed to the drawings, where like reference numerals or characters indicate corresponding or like components. In the drawings:

FIG. 1 is a top view of a cover of a scalp protector according to an embodiment of the present invention;

FIG. 2 is a schematic isometric view of a band of a scalp protector in an extended configuration according to an embodiment of the present invention;

FIG. 3 is a rear isometric view of a scalp protector according to an embodiment of the present invention showing the cover of FIG. 1 fastened to the band of FIG. 2 such that the snapback closure of the band of FIG. 2 is shown;

FIG. 4 is a schematic isometric view of a scalp protector according to an embodiment of the present invention showing the cover of FIG. 1 fastened to the band of FIG. 2 in its fastened configuration;

FIG. 5 is a schematic isometric view of a scalp protector according to an embodiment of the present invention positioned on top of a subject's scalp; and,

FIG. 6 is a side view of a scalp protector according to an embodiment of the present invention showing the fastening of an elongated portion of the cover of FIG. 1 to an anchoring region of the band of FIG. 2 in its fastened configuration;

### DETAILED DESCRIPTION OF THE DRAWINGS

The present invention is not limited in its application to the details of construction and the arrangement of the components set forth in the following description. The invention is capable of other embodiments, or of being practiced or carried out in various ways. Also, it is to be understood that the phraseology and terminology employed herein is for the purpose of description and should not be regarded as limiting.

The present invention provides a scalp protector for use following a hair transplant procedure conducted on a subject. The scalp protector includes a band configured to extend around the periphery of the scalp of the subject, and a cover for covering the surface of the scalp of the subject. The band and the cover are connected using snap fasteners closure.

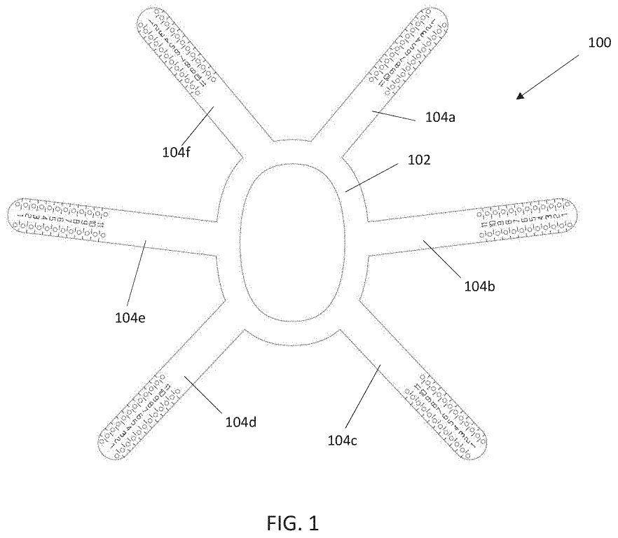

FIG. 1 is a top view of the cover 100. The cover 100 includes a central region 102 and elongated portions 104a-104f extending from the central region 102. Each elongated portion 104a-104f includes one or more sets of a first part of a snap fasteners closure in the form of, for example, a horizontal strip located at its distal portion. The one or more sets of a first part of a snap fasteners closure can be, for example, either a set of socket fasteners or a set of stud fasteners according to two different embodiments of the invention.

In another embodiment, each elongated portion 104a-104f may include a first part of other types of fasteners closures, for example, a first part of a hook and loop fastener, a first part of a magnetic fastener, a first part of a snap-fit fastener, and the like.

The inner surface (the surface facing the scalp of the subject) of central region 102, as well as the inner surface of each elongated portion 104a-104f, may include removable pads or bandages (not shown). The removable pads or bandages contact the subject's scalp so as to absorb secretions, such as blood, pus or sweat secreted from wounds or bruises that may potentially form on the scalp of the subject following a hair transplant procedure.

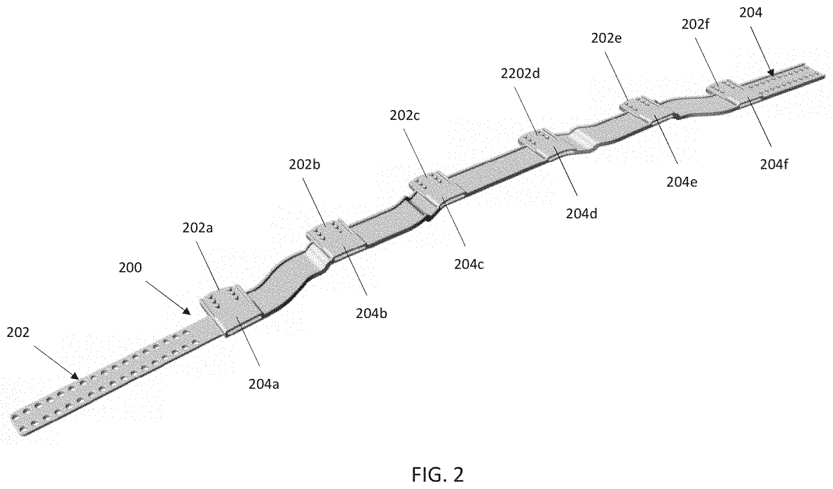

FIG. 2 is a top left view of the band 200 in an extended configuration. At its fastened configuration, the band 200 is shaped to extend, for example, from the subject's forehead to his nape.

The band 200 includes anchoring regions 202a-202f deployed along its length. The anchoring regions 202a-202f are deployed such that each anchoring region aligns with its corresponding elongated portion 104a-104f of cover 100. Each anchoring region 202a-202f includes one or more sets of a complementary part of a snap fasteners closure in the form of a horizontal strip for fastening each of elongated portions 104a-104f to its corresponding anchoring region 202a-202f. The one or more sets of a complementary part of a snap fasteners closure can be, for example, either a set of stud fasteners or a set of socket fasteners according to two different embodiments of the invention.

In another embodiment, each anchoring region 202a-202f may include a complementary part of other types of fasteners closures, for example, a complementary part of a hook and loop fastener, a complementary part of a magnetic fastener, a complementary part of a snap-fit fastener, and the like.

The number of fasteners in each set of the one or more sets of fasteners of anchoring regions 202a-202f can vary from the number of fasteners in each set of the one or more sets of fasteners of elongated portions 104a-104f. This difference enables fastening either the anchoring regions or the elongated portions having one or more sets of fasteners with fewer fasteners to different regions of the one or more sets of complementary fasteners on the corresponding elongated portions or anchoring regions, and by that allows adjusting the distance between the cover 100 and the scalp of the subject.

Anchoring regions 202a-202f further includes securing straps 204a-204f. Each securing strap extends horizontally from one end of the anchoring region to its other end. The securing straps 204a-204f hold each elongated portion 104a-104f attached to its aligned anchoring region 202a-202f.

The inner surface of band 200 (the surface facing the scalp of the subject) may include removable pads or bandages (not shown) contacting the subject's scalp so as to absorb secretions, such as blood, pus or sweat secreted from wounds or bruises that may potentially form on the scalp of the subject following a hair transplant procedure.

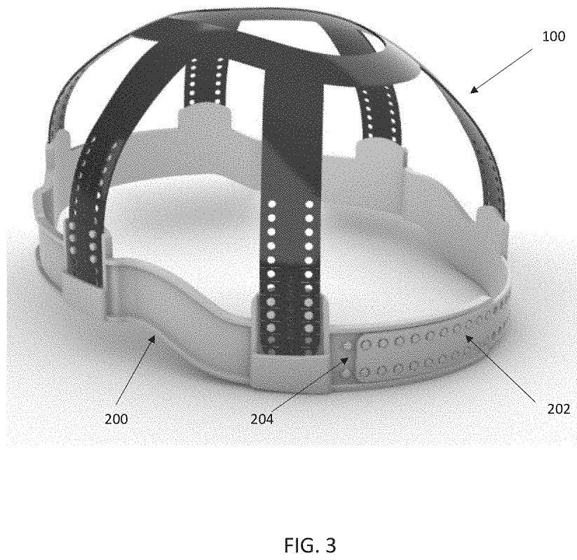

The band 200 further includes one or more sets of a first part of a snapback closure on its proximal end 202, and one or more sets of a complementary part of a snapback closure on its distal end 204 for fastening both ends together around the subject's scalp. The snapback closure enables adjusting the perimeter of band 200 to fit closely to the perimeter of the subject's scalp (best seen in FIG. 3).

The one or more sets of a first part of a snapback closure can be, for example, either a socket fastener or a stud fastener according to two different embodiments of the invention.

In one embodiment, the band 200 is made of a single portion such that, when the band 200 is in its fastened configuration, all sections of band 200 are in contact with the subject's scalp.

In another embodiment, the band 200 consists of a plurality of fragments deployed such that when the band is in its fastened configuration some fragments are in contact with regions of the subject's scalp, such as the forehand and the nape, while other sections of the subject's scalp, such as the temporal recession areas remain unattached to fragments of the band 200.

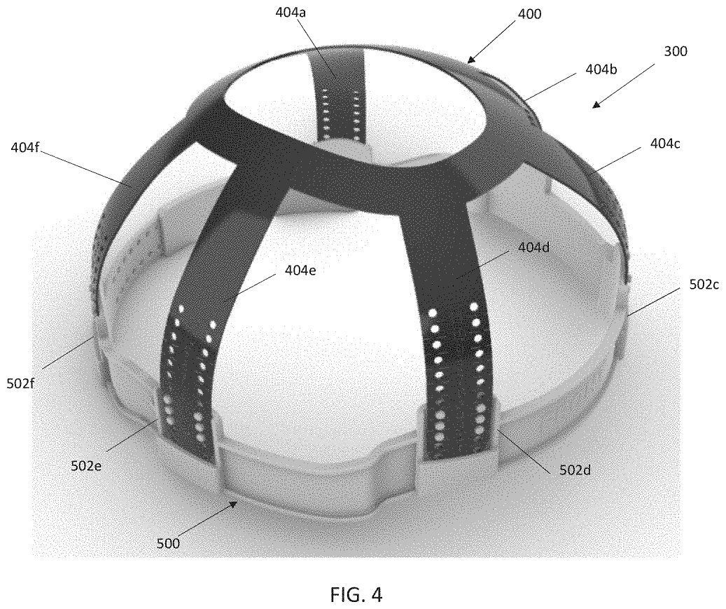

FIG. 4 is a schematic isometric view of the scalp protector 300. The scalp protector 300 includes a cover 400, for example, the cover of FIG. 1 and a band 500, for example, the band of FIG. 2.

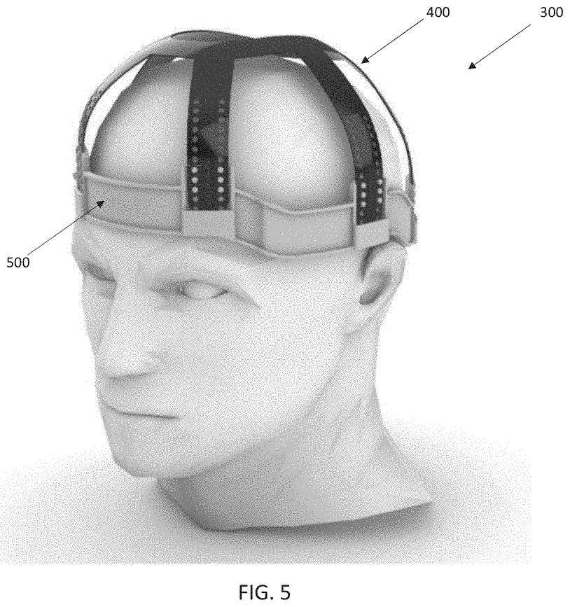

The cover 400 and the band 500 are typically made of a semi-rigid material with somewhat elastic properties, such as silicon or plastic, for deployment around the periphery of a subject's scalp (best seen in FIG. 5). The entire cover 400 and band 500 may be formed from a material having somewhat elastic properties, or specific regions located at one or more areas of the cover and/or band may be elastic, thereby facilitating the fitting of the scalp protector 300 around the subject's scalp.

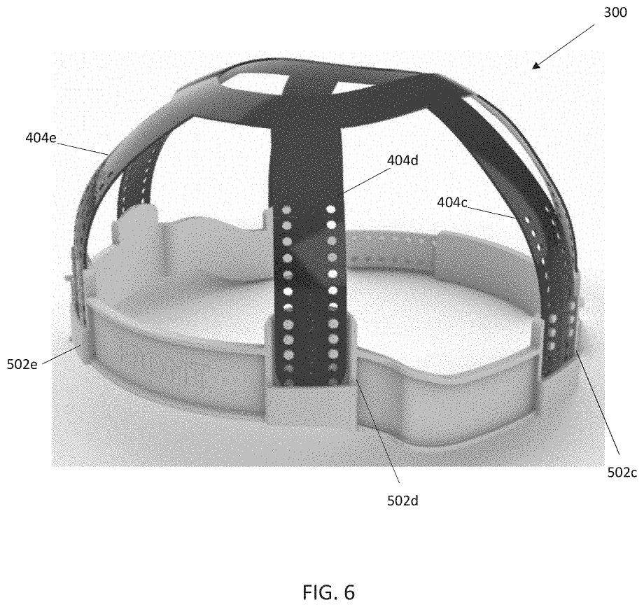

In operation, each elongated portion 404a-404f of cover 400 is fastened to its complementary anchoring region 502a-502f on band 500. The one or more sets of fasteners of the anchoring regions 502a-502f are fastened to a desired area of the one or more sets of fasteners of the elongated portions 404a-404f (best seen in FIG. 6) so as to achieve the preferred distance between the scalp protector 300 and the subject's scalp.

Once the elongated portions 404a-404f are fastened to their corresponding anchoring regions 502a-502f, each elongated portion 404a-404f curves into a scythe-like shape. The scythe-like shape of the elongated portions 404a-404f gives the cover 400 a dome-like shape that extends around the subject's scalp. The dome-like shape covers the surface of a subject's scalp without contacting it.

Following a hair transplant procedure, the scalp protector 300 is laid on top of the subject's scalp allowing the subject to protect his wounded scalp. The scalp protector 300 can then be covered using a headwear, for example, a hat, a cap, a bandana and the like so as to achieve further protection to the subject's scalp and to give the subject's scalp a more aesthetic look.

While the invention has been described with respect to a limited number of embodiments, it will be appreciated that many variations, modifications and other applications of the invention may be made. Therefore, the claimed invention as recited in the claims that follow is not limited to the embodiments described herein.

1. A scalp protector for use following a hair transplant procedure conducted on a subject comprising:
a cover having a central region and elongated portions extending from said central region for covering the surface of the scalp of said subject, each of said elongated portions provides at least one set of a first part of a snap fasteners closure;
a band configured to extend around the periphery of the scalp of said subject, said band includes anchoring regions deployed on said band so as to align with the elongated portions of said cover, each of said anchoring regions providing at least one set of a complementary part of a snap fasteners closure for fastening to said elongated portions;

2. The scalp protector of claim 1, wherein said band includes at least one set of a first part of a snapback closure on its proximal end and at least one set of a complementary part of a snapback closure on its distal end for fastening both ends of said band together.

3. The scalp protector of claim 2, wherein said at least one set of a first part of a snapback closure on the proximal end of said band is a set of stud fasteners and said at least one set of a complementary part of a snapback closure on the distal end of said band is a set of socket fasteners.

4. The scalp protector of claim 2, wherein said at least one set of a first part of a snapback closure on the proximal end of said band is a set of socket fasteners and said at least one set of a complementary part of a snapback closure on the distal end of said band is a set of stud fasteners.

5. The scalp protector of claim 1, wherein said anchoring regions of said band include securing straps shaped to hold said elongated portions of said cover attached to said aligned anchoring regions.

6. The scalp protector of claim 1, wherein said at least one set of a first part of a snap fasteners closure in said anchoring regions is a set of stud fasteners and said at least one set of a complementary part of a snap fasteners closure is a set of socket fasteners.

7. The scalp protector of claim 6, wherein said set of stud fasteners and said set of socket fasteners have uneven numbers of fasteners so as to adjust the distance of said cover from the scalp of said subject.

8. The scalp protector of claim 1, wherein said at least one set of a first part of a snap fasteners closure in said anchoring regions is a set of socket fasteners and said at least one set of a complementary part of a snap fasteners closure is a set of stud fasteners.

9. The scalp protector of claim 8, wherein said set of socket fasteners and said set of stud fasteners have uneven numbers of fasteners so as to adjust the distance of said cover from the scalp of said subject.

10. The scalp protector of claim 1, wherein said band includes removable pads or bandages on the inner surface of said band, said removable pads or bandages shaped so as to contact the subject's scalp.

11. The scalp protector of claim 1, wherein said cover includes removable pads or bandages on the inner surface of said cover, said removable pads or bandages shaped so as to contact the subject's scalp.

12. The scalp protector of claim 1, wherein said band includes a plurality of fragments at different distances in relation to the scalp of said subject.

13. The scalp protector of claim 1, wherein said band is made of a semi rigid material.

14. The scalp protector of claim 13, wherein said band is made of silicone.

15. The scalp protector of claim 1, wherein said cover is made of a semi rigid material.

16. The scalp protector of claim 15, wherein said cover is made of silicone.
